(19) United States
(12) Patent Application Publication
(10) Pub. No.: US 2025/0302684 A1
(43) Pub. Date: Oct. 2, 2025

(54) BEDSORE PREVENTION MATTRESS

(71) Applicant: AIGEM CO., LTD., Gwangju (KR)

(72) Inventor: Young Gu LEE, Seoul (KR)

(73) Assignee: AIGEM CO., LTD., Gwangju (KR)

(21) Appl. No.: 19/233,717

(22) Filed: Jun. 10, 2025

Related U.S. Application Data
(63) Continuation of application No. PCT/KR2023/020431, filed on Dec. 12, 2023.

(30) Foreign Application Priority Data
Dec. 12, 2022 (KR) 10-2022-0174126

(51) Int. Cl. A61G 7/057; A61H 23/02; A61M 21/00

(52) U.S. Cl. CPC A61G 7/05776; A61H 23/02; A61M 2021/0022; A61M 2021/0083

(57) ABSTRACT

The present invention relates to a bedsore prevention mattress and, more specifically, to the bedsore prevention mattress in which a portion of the mattress surface expands and contracts through a pneumatic pump, and which minimizes body contact areas where the patient's weight is concentrated during the expansion and contraction process, thereby reliably preventing the occurrence of bedsores. To this end, the present invention provides the mattress adapted for use on an upper surface of a bed, the mattress comprising: grid-type air cells, each having an independent internal chamber; resonant vibration speakers, each attached inside a corresponding grid-type air cell and configured to generate vibrations to massage an affected area of a patient; and a base positioned below the grid-type air cells and coupled thereto to form an integrated mattress.

### CROSS-REFERENCE TO RELATED APPLICATION

The present application is a Continuation of International Application No. PCT/KR2023/020431 filed on Dec. 12, 2023, which claims priority to Korean patent application number 10-2022-0174126, filed on Dec. 13, 2022, which is incorporated herein by reference in its entirety.

### TECHNICAL FIELD

The present invention relates to a mattress for preventing bedsores, in particular, a bedsore prevention mattress in which only a portion of the mattress surface expands and contracts by means of a pneumatic pump, thereby minimizing the body contact area where the patient's weight is concentrated during the expansion and contraction process to effectively and stably prevent bedsores.

### BACKGROUND

Generally, bedding mattresses are of a type in which a plurality of coil springs are arranged inside to maintain cushioning and shape by means of the elastic force and resilience of the springs.

However, mattresses that use coil springs have drawbacks in that an impact on one part is transmitted to surrounding areas, causing significant vibrations, and since the elastic force of the springs is predetermined during manufacturing, the user cannot adjust the cushioning according to their body conditions.

Moreover, when used over a long period, the elastic force and resilience of the springs deteriorate, making it difficult to maintain cushioning and shape, and generating noise that shortens the product's lifespan.

To address such drawbacks, air mattresses using pneumatic technology have been developed and applied.

### DISCLOSURE OF INVENTION

### Technical Problem

The object of the present invention is to provide the bedsore prevention mattress that solves the aforementioned problems by enabling only a part of the mattress surface to expand and contract through a pneumatic pump and minimizing the contact area where the patient's weight is concentrated during such expansion and contraction to reliably prevent bedsores.

Another object of the present invention is to provide a bedsore prevention mattress in which fold grooves are formed on the upper and lower portions of the mattress so that the upper and lower body parts of the mattress can bend in synchronization with the folding action of the bed.

A further object of the invention is to provide the bedsore prevention mattress equipped with pressure and temperature sensors on the surface to detect changes in the patient's body weight and temperature for enhanced bedsore prevention.

### Solution to the Problem

The present invention achieves the above objectives through the following technical means:
a mattress stacked on the upper surface of a bed, comprising:
a grid-type air cell, each having an independent internal chamber;
a resonant vibration speaker attached inside each of the grid-type air cell, configured to generate vibrations to massage the affected area of a patient; and
a base located below the grid-type air cell and coupled thereto to form a unified mattress structure;
wherein the resonant vibration speaker generates vibrations synchronized with music to notify the patient of wake-up or medication times;
wherein the resonant vibration speaker is attached to an upper portion inside the plurality of grid-type air cell, and a corresponding pocket is formed on the surface of the base at the same position as the resonant vibration speaker;
wherein the resonant vibration speaker moves up and down within the pocket as the plurality of grid-type air cell expands and contracts vertically, thereby preventing the formation of bedsores;
wherein the independent grid-type air cell expand and contract via a pneumatic pump to reduce pressure caused by the patient's body weight and thereby prevent bedsores;
wherein pressure sensors and temperature sensors are arranged in a cross pattern within the grid-type air cell to detect changes in the patient's weight and temperature;
and wherein fold grooves are formed at predetermined intervals on the upper portion of the grid-type air cell and the lower portion of the base, allowing the mattress to fold in synchronization with the elevation of the upper and lower portions of the bed.

### Effects of the Invention

According to the present invention, only part of the mattress surface expands and contracts by means of a pneumatic pump, effectively minimizing body contact areas with concentrated pressure, thereby reliably preventing bedsores.

Also, the inclusion of pressure and temperature sensors allows for detection of body weight and temperature changes, enabling more efficient management of bedsore risks.

Furthermore, fold grooves formed on the mattress facilitate synchronized bending of the mattress when the upper and lower portions of the bed are elevated.

### BRIEF DESCRIPTION OF DRAWINGS

FIG. 1 is a perspective view of the bedsore prevention mattress according to the present invention.

FIG. 2 is an enlarged perspective view of the essential parts of the mattress.

FIG. 3 is an exploded view showing the separation of the grid-type air cell and base.

FIG. 4 is a partial cut-away view showing the resonant vibration speaker and pocket.

FIG. 5 is a perspective view showing the mattress placed on top of a bed.

FIG. 6 is a side view showing the folded state of the mattress with fold grooves.

### DETAILED DESCRIPTION

Although various modifications and implementations are possible within the scope of the present invention, only specific embodiments are described in detail below. The scope of the invention is not limited to these embodiments but includes all equivalent modifications and substitutions falling within the spirit of the invention.

To avoid obscuring the essential aspects of the invention, detailed descriptions of well-known technologies, such as the use of a pneumatic pump for expansion/contraction, sound-driven vibration by the resonant vibration speaker, and electrical control of the sensors, are omitted.

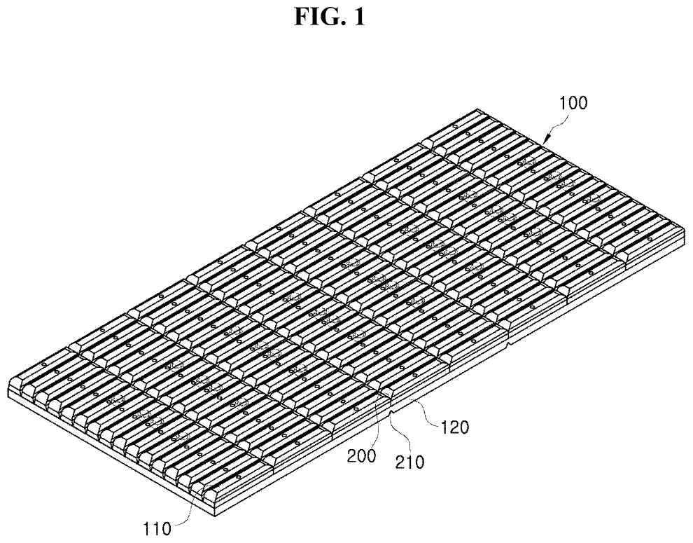

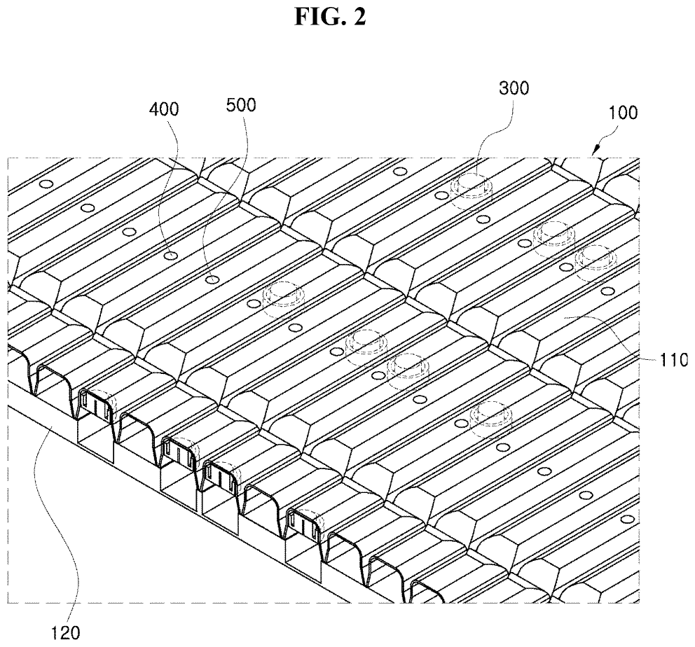

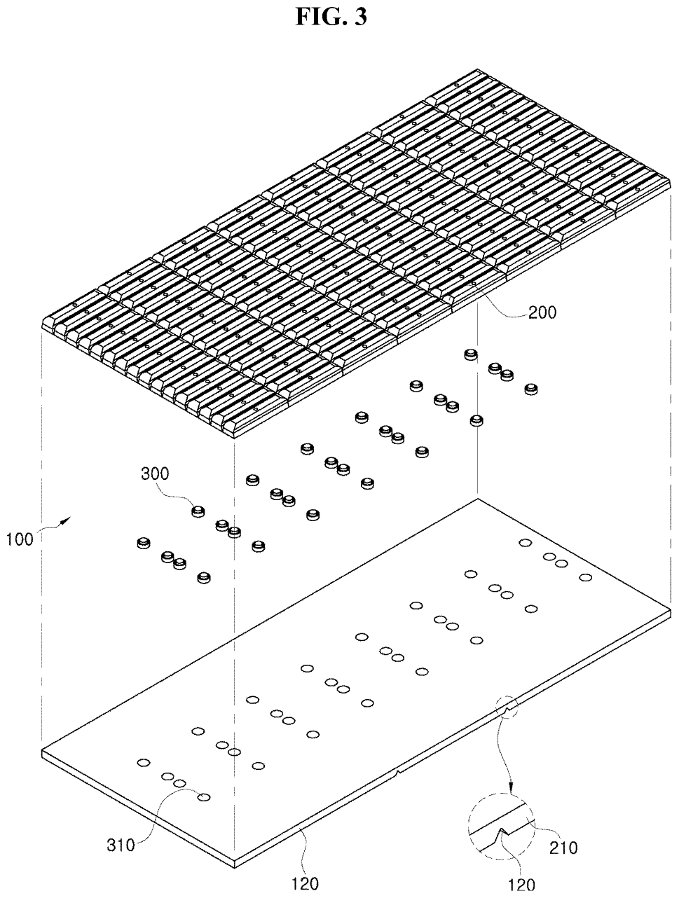

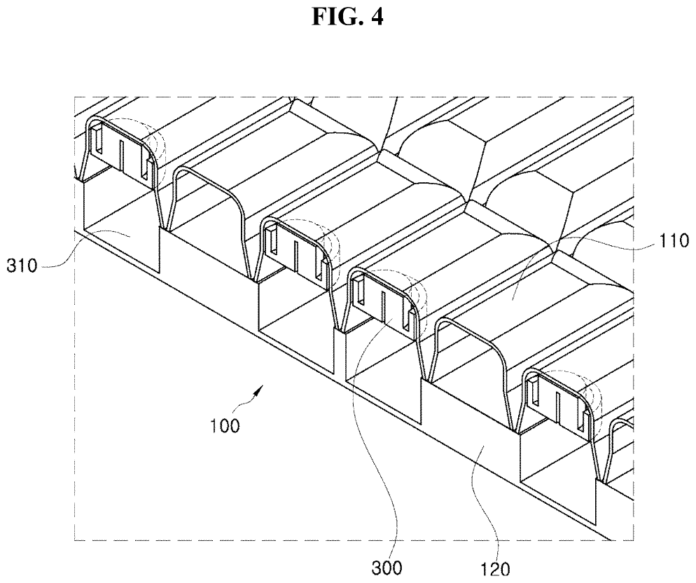

As illustrated in FIGS. 1 through 6, the mattress (100) is configured for placement on the top surface of a bed (10) and includes grid-type air cell (110), each having an independent internal chamber; a resonant vibration speaker (300) attached inside each air cell for massaging the patient's body; and a base (120) coupled to the lower part of the grid-type air cell.

The resonant vibration speaker (300) generates vibrations in synchronization with music to notify the patient of wake-up or medication times or may provide massage by vibration.

Additionally, the resonant vibration speaker (300) is positioned inside the upper portion of the grid-type air cell (110), and corresponding pockets (310) are formed in the base (120) at positions directly beneath the resonant vibration speakers, allowing vertical movement of the resonant vibration speakers during the expansion and contraction of the grid-type air cell to minimize body contact pressure and prevent bedsores.

The expansion and contraction of the grid-type air cell (110) occur in alternating cycles.

Each air cell is equipped with a pressure sensor (400) and a temperature sensor (500) to detect the patient's body weight and temperature changes. The sensors are arranged in a cross-pattern across the grid-type air cell. The pressure sensor (400) detects weight distribution and pressure concentration, while the temperature sensor (500) detects temperature variations caused by sustained pressure. This allows effective monitoring and prevention of bedsores by identifying prolonged pressure points.

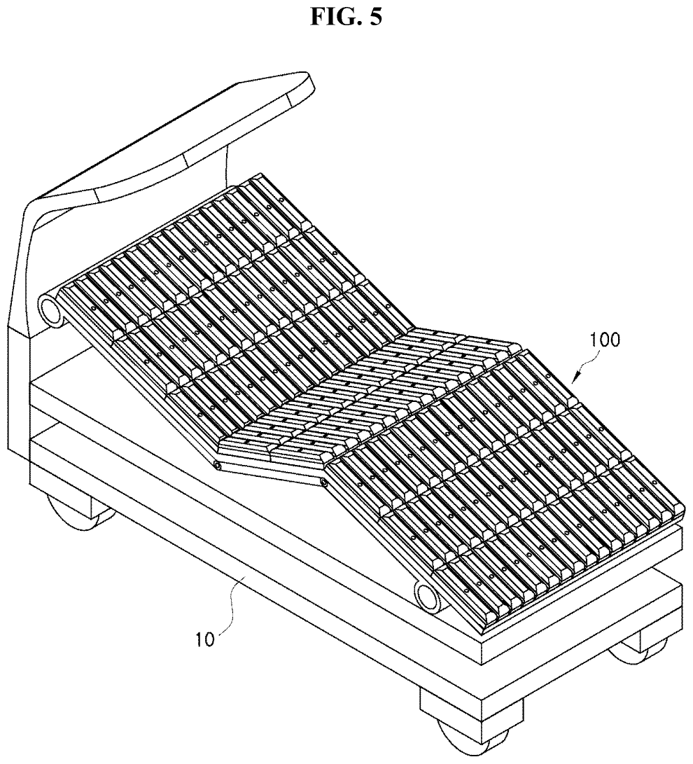

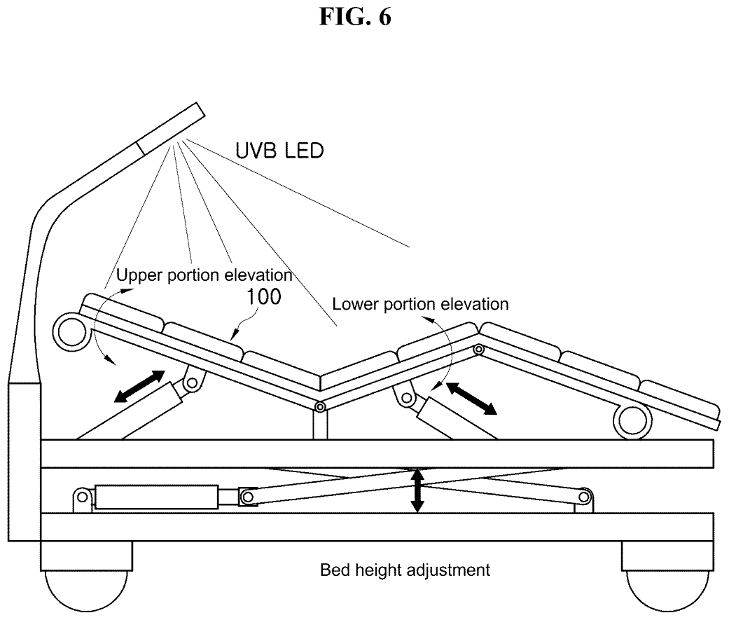

Fold grooves (200, 210) are formed at regular intervals on the upper surface of the grid-type air cell (110) and the lower surface of the base (120). As illustrated in FIGS. 5 and 6, these grooves allow the mattress (100) to bend in synchronization with the bed's movement when elevating the upper and lower sections.

The above description is illustrative of the technical concept of the present invention and not intended to limit the invention. Various modifications and substitutions are possible without departing from the scope of the invention by those skilled in the art. The scope of protection shall be determined by the claims below, and all technical equivalents shall be construed as being within the scope of the present invention.

1. A bedsore prevention mattress adapted for use on an upper surface of a bed, comprising:
a grid-type air cell, each having an independent internal chamber;
a resonant vibration speaker attached inside the grid-type air cell, configured to generate vibrations to massage an affected area of a patient; and
a base positioned below the grid-type air cell and coupled to the grid-type air cell to form an integrated mattress.

2. The bedsore prevention mattress of claim 1,
wherein the resonant vibration speaker generates vibrations synchronized with music to notify the patient of wake-up or medication times.

3. The bedsore prevention mattress of claim 1,
wherein the resonant vibration speaker is attached to an upper portion inside the grid-type air cell, and a pocket is formed on a surface of the base at a position corresponding to the resonant vibration speaker.

4. The bedsore prevention mattress of claim 3,
wherein the resonant vibration speaker moves vertically within the pocket as the grid-type air cell expands and contracts to prevent bedsores.

5. The bedsore prevention mattress of claim 1,
wherein each independent grid-type air cell expands and contracts vertically by means of a pneumatic pump to reduce pressure caused by the patient's body weight and thereby prevent bedsores.

6. The bedsore prevention mattress of claim 1,
wherein pressure sensors and temperature sensors are arranged in a cross pattern within the grid-type air cell to detect changes in the patient's body weight and temperature.

7. The bedsore prevention mattress of claim 1,
wherein fold grooves are formed at regular intervals on an upper portion of the grid-type air cell and on a lower portion of the base, allowing the mattress to fold in synchronization with elevation of the upper and lower parts of the bed.
